(19) United States
(12) Patent Application Publication
(10) Pub. No.: US 2025/0120917 A1
(43) Pub. Date: Apr. 17, 2025

(54) COMPOSITION FOR FORMULATION, FORMULATION AND METHOD FOR MANUFACTURING COMPOSITION FOR FORMULATION

(71) Applicant: SUMITOMO SEIKA CHEMICALS CO., LTD., Kako-gun, Hyogo (JP)

(72) Inventors: Motoharu FUJISAKI, Himeji-shi (JP); Akira KIMURA, Himeji-shi (JP)

(73) Assignee: SUMITOMO SEIKA CHEMICALS CO., LTD., Kako-gun, Hyogo (JP)

(21) Appl. No.: 18/834,125

(22) PCT Filed: Jan. 27, 2023
(86) PCT No.: PCT/JP2023/002610

§ 371 Date: Jul. 29, 2024

(30) Foreign Application Priority Data
Jan. 31, 2022 (JP) 2022-013786

(51) Int. Cl. A61K 9/16; A61K 31/167

(52) U.S. Cl. CPC A61K 9/1629; A61K 9/1694; A61K 31/167

(57) ABSTRACT

A preparation composition that is easy to produce and enables a preparation with high hardness to be easily obtained, a preparation comprising the composition, and a method for producing the preparation composition are provided. The preparation composition comprises particles containing an active ingredient and a binder. The particles contain the active ingredient in an amount of 80 mass % or more based on the total mass of the active ingredient and the binder, and the coverage of the binder at the surface of the particles is 20% or more. The method for producing the preparation composition includes step 1 of kneading and granulating a mixture containing an active ingredient and a binder in barrels by a twin-screw extrusion method, the temperature in each barrel being 130° C. or less, the binder having a mass average molecular weight of 1,000,000 g/mol or less, and the mixture containing the active ingredient in an amount of 80 mass % or more based on the total mass of the active ingredient and the binder.

### TECHNICAL FIELD

The present invention relates to a preparation composition, a preparation, and a method for producing the preparation composition.

### BACKGROUND ART

It is known that preparations that release drugs, such as controlled drug release preparations, can be obtained by various methods. For example, in a known method, a preparation is obtained by mixing a raw material containing an active ingredient and a binder to prepare a mixture, granulating the mixture to form particles (granules), and forming tablets using the granules.

As methods for obtaining such granules, a wet granulation method, a dry granulation method, etc. are known. For example, Patent Literature 1 proposes a method in which a raw material containing an active ingredient and a binder is kneaded and granulated with a twin-screw extruder. This method requires less energy during production and can be applied to raw materials containing drugs that are sensitive to moisture.

### CITATION LIST

### Patent Literature

PTL 1: JP2008-540540A

### SUMMARY OF INVENTION

### Technical Problem

However, conventional particles (granules) containing an active ingredient and a binder do not have sufficient hardness, and thus tend to be unsuitable as a preparation. Methods such as changing production conditions of twin-screw extrusion etc. and adjusting the amount of binder can be considered to improve hardness; however, these are problematic in that the granulation properties may decrease, making it difficult to obtain a preparation.

The present invention has been made in view of the above, and an object of the present invention is to provide a preparation composition that is easy to produce and enables a preparation with high hardness to be easily obtained, a preparation comprising the composition, and a method for producing the preparation composition.

### Solution to Problem

The present inventors conducted extensive research to achieve the above object, and found that the object can be achieved by setting the amounts of an active ingredient and a binder to predetermined amounts and setting the coverage of the binder to a predetermined percentage. Thus, the present invention has been accomplished.

Specifically, the present invention encompasses, for example, the subject matter of the following items.

### Item 1

A preparation composition comprising particles containing an active ingredient and a binder,
the particles containing the active ingredient in an amount of 80 mass % or more based on the total mass of the active ingredient and the binder, and
the coverage of the binder at the surface of the particles being 20% or more.

### Item 2

The preparation composition according to Item 1, wherein the binder is a water-soluble polymer.

### Item 3

The preparation composition according to Item 1 or 2, wherein the binder is at least one member selected from the group consisting of polyethylene oxide, hydroxypropyl cellulose, hydroxypropyl methylcellulose, polyvinylpyrrolidone, polymethacrylate, copolymers of N-vinylpyrrolidone and vinyl acetate, and copolymers of N-vinylcaprolactam, vinyl acetate, and ethylene glycol.

### Item 4

The preparation composition according to any one of Items 1 to 3, wherein the binder has a mass average molecular weight of 1000000 g/mol or less.

### Item 5

The preparation composition according to any one of Items 1 to 4, wherein the binder is polyethylene oxide having a mass average molecular weight of 50000 g/mol or more and 1000000 g/mol or less.

### Item 6

A preparation comprising the preparation composition according to any one of Items 1 to 5.

### Item 7

A method for producing the preparation composition according to any one of Items 1 to 5, the method comprising step 1 of kneading and granulating a mixture containing an active ingredient and a binder in barrels by a twin-screw extrusion method,
the temperature in each barrel being 130° C. or less,
the binder having a mass average molecular weight of 1000000 g/mol or less, and
the mixture containing the active ingredient in an amount of 80 mass % or more based on the total mass of the active ingredient and the binder.

### Advantageous Effects of Invention

The preparation composition of the present invention is easy to produce and enables a preparation with high hardness to be easily obtained.

### BRIEF DESCRIPTION OF DRAWINGS

FIG. 1 is a schematic view showing an example of a screw used in the production of the preparation composition of the present invention.

FIG. 2 is a schematic view showing an example of a screw used in Examples.

### DESCRIPTION OF EMBODIMENTS

Embodiments of the present invention are described in detail below. In the present specification, the terms “comprising” and “containing” include the concepts of “comprising,” “containing,” “consisting essentially of,” and “consisting of.”

In the numerical range described in stages in the present specification, the upper or lower limit of the numerical range at one stage can be optionally combined with the upper or lower limit of the numerical range at another stage. In the numerical range described in the present specification, the upper or lower limit of the numerical range may be replaced with a value shown in the Examples or a value that can be uniquely derived from the Examples. Further, in the present specification, the numerical values connected by “to” mean the numerical range including the numerical values before and after “to” as the lower limit and the upper limit.

### 1. Preparation Composition

The preparation composition of the present invention comprises particles containing an active ingredient and a binder, the particles contain the active ingredient in an amount of 80 mass % or more based on the total mass of the active ingredient and the binder, and the coverage of the binder at the surface of the particles is 20% or more.

The preparation composition of the present invention is easy to produce, enables a preparation having high hardness to be easily obtained, and is suitable as a raw material for producing a preparation.

The type of active ingredient is not particularly limited, and a wide range of known active ingredients can be used. Examples of active ingredients include drugs such as acetaminophen, which is used as, for example, an antipyretic or a painkiller.

The active ingredient can be produced by a known method or can be obtained from, for example, a commercially available product.

The type of binder is not particularly limited, and, for example, a wide range of binders used in preparations can be used in the present invention. In particular, various polymer materials having a mass average molecular weight of 1000000 g/mol or less can be used as the binder. In this case, the coverage can be easily adjusted to a desired range, and a preparation with high hardness can be easily prepared. The various polymer materials having a mass average molecular weight of 1000000 g/mol or less may be homopolymers or copolymers.

The binder is preferably a water-soluble polymer in terms of excellent solubility in water. Examples of water-soluble polymers include polyvinylpyrrolidone, copolymers of N-vinylpyrrolidone and vinyl acetate, copolymers of N-vinylcaprolactam, vinyl acetate, and ethylene glycol, copolymers of N-vinylpyrrolidone and vinyl propionate, cellulose esters, cellulose ethers, hydroxyalkyl celluloses (e.g., hydroxypropyl cellulose and hydroxypropyl methylcellulose), cellulose phthalate, cellulose succinate, polyalkylene oxides (e.g., polyethylene oxide and polypropylene oxide), copolymers of ethylene oxide and propylene oxide, polyacrylate, polymethacrylate, polyacrylamide, vinyl acetate polymers, polyvinyl alcohol, oligosaccharides, polysaccharides, and the like.

The binder is more preferably at least one member selected from the group consisting of polyethylene oxide, hydroxypropyl cellulose, hydroxypropyl methylcellulose, polyvinylpyrrolidone, polymethacrylate, copolymers of N-vinylpyrrolidone and vinyl acetate, and copolymers of N-vinylcaprolactam, vinyl acetate, and ethylene glycol. In this case, the particles (also referred to as “granules”) described above are easily formed (i.e., the granulation properties are excellent); thus, the preparation composition can be easily produced, and a preparation with high hardness can be easily prepared.

In terms of particularly excellent granulation properties at low temperatures and ease of preparing a preparation with higher hardness, the binder is more preferably at least one member selected from the group consisting of polyethylene oxide and hydroxypropyl cellulose, and particularly preferably polyethylene oxide. In particular, if the binder is polyethylene oxide, granules (particles) with excellent formability can be easily obtained even when kneading is performed at lower temperatures; thus, the preparation composition can be easily produced, and a preparation with high hardness can be easily prepared.

The mass average molecular weight of the binder is preferably 50000 g/mol or more and 1000000 g/mol or less. In this case, granulation becomes easy in the production of the preparation composition, and a preparation with high hardness can be easily prepared. The mass average molecular weight of the binder is more preferably 80000 g/mol or more, and even more preferably 100000 g/mol or more, and is also more preferably 850000 g/mol or less, even more preferably 750000 g/mol or less, and particularly preferably 300000 g/mol or less.

The binder is preferably polyethylene oxide having a mass average molecular weight of 50000 g/mol or more and 1000000 g/mol or less, and particularly preferably 100000 g/mol or more and 300000 g/mol or less.

The binder can be produced by a known method or can be obtained from, for example, a commercially available product. For example, when the binder is a polyalkylene oxide, the polyalkylene oxide can be produced by, for example, polymerization reaction of an alkylene oxide in the presence of a catalyst.

The preparation composition of the present invention can comprise particles containing the active ingredient and the binder as the main component. For example, the preparation composition of the present invention can comprise particles containing the active ingredient and the binder in an amount of 50 mass % or more, preferably 80 mass % or more, more preferably 90 mass % or more, even more preferably 95 mass % or more, and particularly preferably 99 mass % or more. The preparation composition of the present invention may consist of particles containing the active ingredient and the binder.

The particles may contain one active ingredient or two or more active ingredients. Similarly, the particles may contain one binder or two or more binders.

The particles may contain other components as long as they contain the active ingredient and the binder. The particles may also consist of the active ingredient and the binder. Examples of other components include various additives contained in known preparations, such as fillers, excipients, diluents, lubricants, dyes, and pigments. Specific examples include silica, such as Aerosil.

When the particles contain other components (e.g., additives described above), the content of the other components is preferably 10 mass % or less, more preferably 5 mass % or less, even more preferably 1 mass % or less, and particularly preferably 0.5 mass % or less, based on the total mass of the active ingredient and the binder.

The particles contain the active ingredient in an amount of 80 mass % or more based on the total mass of the active ingredient and the binder (i.e., the particles contain the binder in an amount of 20 mass % or less based on the total mass of the active ingredient and the binder). This makes it easy to produce the preparation composition and also makes it easy to prepare a preparation with high hardness. If the content of the binder in the particles exceeds 20 mass % based on the total mass of the active ingredient and the binder, it is difficult to obtain a preparation from the preparation composition, and, for example, tablets are likely to partially chip. Even if a preparation can be obtained, the preparation is less likely to have high hardness. From the viewpoint of excellent granulation properties during production, the content of the binder in the particles is preferably 5 mass, or more, and more preferably 7.5 mass % or more, based on the total mass of the filler, the active ingredient, and the binder.

The granules more preferably contain the active ingredient in an amount of 85 mass % or more based on the total mass of the active ingredient and the binder. The particles also preferably contain the active ingredient in an amount of 99 mass % or less, more preferably 97 mass % or less, and particularly preferably 95 mass % or less, based on the total mass of the active ingredient and the binder.

The form of the particles is not particularly limited, and may be, for example, spherical, ellipsoidal, or amorphous. The particles may also be agglomerated particles formed by agglomeration of multiple particles.

The size of the particles is not particularly limited, and, for example, can be suitably adjusted according to a desired preparation. For example, the particles preferably have a median particle size of 100 to 500 μm.

As described above, the coverage of the binder at the surface of the particles is 20% or more. This enables a preparation with high hardness to be prepared.

In the present invention, the coverage can be measured by an ATR method (attenuated total reflection) using particles as a measurement sample. Specifically, the coverage can be measured by the following method. The same active ingredient and the same binder as the active ingredient and the binder contained in a measurement sample are mixed in different arbitrary mass ratios to prepare six mixtures. The absorbance of each mixture is measured using a Fourier transform infrared spectrometer (JASCO Corporation), and a calibration curve is created from the ratio of the absorbance in an absorption band characteristic of the active ingredient and the absorbance in an absorption band characteristic of the binder. For example, when the active ingredient is acetaminophen, and the binder is polyethylene oxide, the characteristic absorption bands are present at 1650 cm⁻¹ and 2880 cm⁻¹. Thus, a calibration curve is created from the ratio of the absorbances in them. Subsequently, the absorbance of the particles as the measurement sample is also measured using a Fourier transform infrared spectrometer (JASCO Corporation), and the proportion of the binder present relative to the active ingredient and the binder is calculated by substituting the measured absorbance into the function for the calibration curve. This proportion is defined as the coverage.

The coverage of the binder at the surface of the particles is preferably 20% or more, more preferably 25% or more, and even more preferably 30% or more. The coverage of the binder at the surface of the particles is, for example, preferably 70% or less, and more preferably 60% or less.

When the content of the binder in the particles is P mass %, and the coverage is C %, it is preferred that P<C. This means that the binder is distributed more near the surface of the particles than inside the particles, which makes it easy for the preparation composition of the present invention to form a preparation with higher hardness. In short, when the concentration of the binder near the surface of the particles is higher than that in the interior of the particles, a preparation with higher hardness can be easily prepared using the preparation composition of the present invention.

The value of C is preferably 1.5 times or more, more preferably 2 times or more, even more preferably 2.5 times or more, and particularly preferably 3 times or more larger than the value of P. The value of C is also preferably not greater than 9.5 times, and more preferably not greater than 8 times the value of P.

The content P (mass %) of the binder in the particles can be quantified by the following method using gel permeation chromatography. Four aqueous solutions of the particles with different arbitrary binder concentrations are prepared. Each peak area is measured by gel permeation chromatography, and a calibration curve of the binder concentration and peak area is created. Subsequently, an aqueous solution of particles as a measurement sample is prepared, and the peak area is measured to calculate the concentration of the binder in the aqueous solution from the calibration curve. From the ratio of the mass of the binder in the aqueous solution calculated from the binder concentration obtained and the mass of the particles used to prepare the aqueous solution, the content P of the binder can be calculated. The content P of the binder measured by this method can be regarded as corresponding to the content of the binder in the mixture described below used during production.

Since the preparation composition of the present invention contains the particles, a preparation (e.g., tablet) prepared using the composition has high hardness, such as being able to have a compression strength of 2 MPa or more.

In particular, when the particles contained in the preparation composition of the present invention have a structure in which the binder concentration near the surface is higher than that in the interior, as described above, even if the overall concentration of the active ingredient is high, the binder is distributed more at the surface; thus, despite the high concentration of the active ingredient, high hardness is maintained due to the presence of the binder at a high concentration at the surface. Thus, the preparation composition of the present invention enables a preparation having high hardness and a high active ingredient concentration to be prepared, and for example, the efficacy of the active ingredient is easily exhibited even when the preparation is taken in a small amount.

The preparation composition of the present invention may contain other components as long as it contains the particles, as described above. The preparation composition of the present invention may contain a separate binder independent of the particles and may also contain a separate active ingredient. As other components, various components can be used as long as the effects of the invention are not impaired. Examples include a wide range of components that can be contained in conventional preparation compositions. The preparation composition of the present invention may consist of the particles.

### 2. Method for Producing Preparation Composition

The method for producing the preparation composition of the present invention is not particularly limited, and, for example, a wide range of methods for producing known preparation compositions can be used. In particular, as described below, it is preferable to prepare the preparation composition through step 1 in which the particles are obtained by a twin-screw extrusion method. In this case, the coverage can be easily adjusted to a desired range, and particles in which the binder is distributed more at the surface can be easily formed.

The method for producing the preparation composition of the present invention can comprise, for example, step 1 of kneading and granulating a mixture containing an active ingredient and an binder in barrels by a twin-screw extrusion method. In the production method comprising step 1, the temperature in each barrel is 130° C. or less, the binder has a mass average molecular weight of 1000000 g/mol or less, and the mixture contains the active ingredient in an amount of 80 mass % or more based on the total mass of the active ingredient and the binder.

In step 1, a mixture containing an active ingredient and a binder can be used as a raw material. The active ingredient and binder contained in the raw material are the same as the active ingredient and binder contained in the preparation composition of the present invention described above. Thus, various polymer materials having a mass average molecular weight of 1000000 g/mol or less can be used as the binder. Use of a polymer having a mass average molecular weight of 1000000 g/mol or less as the binder allows for production of a preparation composition that enables a preparation with high hardness to be prepared even though the temperature in each barrel is low (130° C. or less).

The binder is more preferably at least one member selected from the group consisting of polyethylene oxide, hydroxypropyl cellulose, hydroxypropyl methylcellulose, polyvinylpyrrolidone, polymethacrylate, copolymers of N-vinylpyrrolidone and vinyl acetate, and copolymers of N-vinylcaprolactam, vinyl acetate, and ethylene glycol. In this case, granulation for the preparation composition can be easily performed (i.e., the granulation properties are excellent) even when the temperature in each barrel is low.

From the viewpoint of particularly excellent granulation properties, the binder is preferably polyethylene oxide having a mass average molecular weight of 50000 g/mol or more and 1000000 g/mol or less, and particularly preferably polyethylene oxide having a mass average molecular weight of 100000 g/mol or more and 300000 g/mol or less.

The binder used as a raw material can be produced by a known method or can be obtained from, for example, a commercially available product. For example, when the binder is a polyalkylene oxide, the polyalkylene oxide can be produced by, for example, polymerization reaction of an alkylene oxide in the presence of a catalyst.

The raw material used in the present invention may contain one active ingredient or two or more active ingredients. Similarly, the raw material used in the present invention may contain one binder or two or more binders.

The raw material used in the method for producing the preparation composition of the present invention may contain other components as long as it contains a mixture containing the active ingredient and the binder. The raw material may consist of the mixture (i.e., the active ingredient and the binder).

Examples of other components include various additives contained in known preparations, such as fillers, excipients, diluents, lubricants, dyes, and pigments. Specific examples include silica, such as Aerosil.

When the raw material contains other components (e.g., additives described above) in addition to the mixture, the content of the other components is preferably 10 mass % or less, more preferably 5 mass % or less, even more preferably 1 mass % or less, and particularly preferably 0.5 mass % or less, based on the total mass of the active ingredient and the binder.

The mixture contained in the raw material contains the active ingredient in an amount of 80 mass % or more based on the total mass of the active ingredient and the binder (i.e., the mixture contains the binder in an amount of 20 mass % or less based on the total mass of the active ingredient and the binder). This makes it easy to perform granulation for the preparation composition even when the temperature in each barrel is low, and also makes it easy to prepare a preparation with high hardness. If the content of the binder in the mixture exceeds 20 mass % based on the total mass of the active ingredient and the binder, it is difficult to obtain a preparation from the preparation composition, and, for example, tablets are likely to partially chip. Even if a preparation can be obtained, the preparation is less likely to have high hardness. From the viewpoint of excellent granulation properties, the content of the binder in the mixture is preferably 5 mass % or more, and more preferably 7.5 mass % or more, based on the total mass of the active ingredient and the binder.

The mixture more preferably contains the active ingredient in an amount of 85 mass % or more based on the total mass of the active ingredient and the binder. The mixture also preferably contains the active ingredient in an amount of 99 mass % or less, more preferably 97 mass % or less, and particularly preferably 95 mass % or less, based on the total mass of the active ingredient and the binder.

The method for preparing the raw material containing the mixture is not particularly limited. For example, the raw material containing the mixture can be prepared by mixing the active ingredient and the binder in predetermined proportions. In preparing the raw material, components other than the active ingredient and the binder can be mixed into the raw material as necessary. The raw material containing the mixture can be prepared by, for example, a mixing means using a known mixer.

### Step 1

Step 1 is the step of kneading and granulating the raw material containing the mixture in barrels by a twin-screw extrusion method. Step 1 enables obtaining granules containing the active ingredient and the binder.

In step 1, the raw material is kneaded and granulated using a twin-screw extruder. As the twin-screw extruder, for example, a wide range of known twin-screw extruders can be used. The type of screw of the twin-screw extruder used in step 1 is not particularly limited, and a wide range of known screws can be used.

In general, screws include a pre-conditioning part (also referred to as a “conveying part”), an agglomeration part (also referred to as a “kneading part”), and a breakage part (also referred to as a “pulverizing part”). The screws used in the present invention preferably include at least an agglomeration part (kneading part), and more preferably include a pre-conditioning part (conveying part), an agglomeration part (kneading part), and a breakage part (pulverizing part). When the screws do not include a breakage part (pulverizing part), it is desirable to perform pulverization after twin-screw extrusion.

In particular, in the present invention, it is preferred that the kneading part is composed of kneading screws and that the rest is composed of screws referred to as “full flight.” In this case, granulation for the preparation composition can be easily performed even when the temperature in each barrel is low, and a preparation with high hardness can be easily prepared.

The kneading part preferably accounts for 15% to 30% of the total screw length. In this case, granulation for the preparation composition can be easily performed even when the temperature in each barrel is low, and a preparation with high hardness can be easily prepared.

The kneading part is preferably placed at any position within the range of 0% to 90% when the tip side (granule outlet side) of both ends of the screws is taken as 0%, and the rear end side (raw material feeding side or raw material inlet side) is taken as 100%. Here, the tip side of the screws means the downstream side in the flow direction of the raw material, and the rear end side of the screws means the upstream side in the flow direction of the raw material.

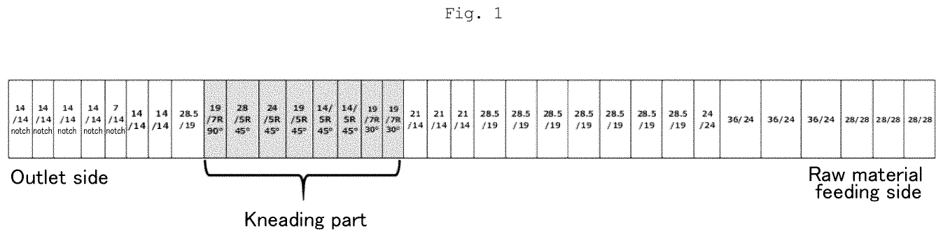

In particular, in the present invention, the screw shown in FIG. 1 is suitably used. In this case, granulation for the preparation composition can be easily performed even when the temperature in each barrel is low, and a preparation with high hardness can be easily prepared.

FIG. 1 is a schematic view of a screw that can be suitably used in the method for producing the preparation composition of the present invention. In the screw shown in FIG. 1, for example, there is the notation “36/24,” in which “36” means the length (mm) of each unit of the screw, “24” means the length (mm) that the blade advances when the screw makes one revolution, which is the “lead.” There is also the notation “28/5R45°,” in which “28” means the length (mm) of each unit of the screw, “5” means the number of kneading disks, “45°” means the angle of the kneading disks, and “R” means that the kneading disks are attached so as to be right-handed. In the notation “7/14 notch,” “7” means the length (mm) of each unit of the screw, “14” means the length (mm) that the blade advances when the screw makes one revolution, which is the “lead,” and “notch” means a notched screw. In FIG. 1, even if there is no “R” notation, it means that the screw is right-handed.

In step 1, the conditions for kneading the raw material in the barrels in the extruder are not particularly limited as long as the temperature in each barrel is 130° C. or less. By kneading the raw material in barrels in which the temperature is 130° C. or less, granulation for the preparation composition can be performed, and a preparation with high hardness can be prepared. Kneading the raw material in barrels in which the temperature is 130° C. or less can also suppress the decomposition of the active ingredient, i.e., a drug. Usually, a plurality of barrels are used. Thus, in the present invention, the temperature in each barrel is set to 130° C. or less.

In step 1, the temperature in each barrel is preferably 120° C. or less, more preferably 110° C. or less, even more preferably 100° C. or less, and particularly preferably 90° C. or less. When a polyalkylene oxide, especially polyethylene oxide, is used as the binder, the granulation properties are excellent even at 90° C. or less, and a preparation with higher hardness can be prepared.

In step 1, the temperature in each barrel is not particularly limited, and is, for example, 20° C. or more, preferably 30° C. or more, and more preferably 40° C. or more.

In the twin-screw extrusion method, the screw speed is not particularly limited and can be, for example, the same as in a method for producing a preparation composition by a known twin-screw extrusion method. For example, the screw speed may be 50 to 500 rpm (the same for commercial screws).

By subjecting the raw material containing the mixture to twin-screw extrusion, particles (granules) containing the active ingredient and the binder can be obtained. The resulting granules may be pulverized as necessary. The pulverization method is not particularly limited. For example, the granules can be pulverized using a known pulverization means. The granules or pulverized granules may be classified as necessary.

### 3. Preparation

The preparation of the present invention comprises the preparation composition of the present invention described above. The dosage form of the preparation is not particularly limited, and is preferably a tablet.

The preparation of the present invention can be obtained by, for example, compressing the preparation composition of the present invention. In this case, the preparation is a tablet. The tablet compression method is not particularly limited, and, for example, can be performed using a known tableting machine.

Since the preparation of the present invention is formed from the preparation composition, the preparation has high hardness, such as being able to have a compression strength of 2 MPa or more. Further, since the concentration of the active ingredient contained in the preparation is high, the preparation is particularly effective as a preparation; for example, the efficacy of the active ingredient is easily exhibited even when the preparation is taken in a small amount.

The features (properties, structures, functions, etc.) described in the embodiments of the present disclosure may be combined in any manner to specify the subject matter included in the present disclosure. That is, this disclosure includes all of the subject matter comprising any combination of the combinable features described herein.

### EXAMPLES

The present invention is described in more detail below with reference to Examples; however, the present invention is not limited to these Examples.

### Example 1

Acetaminophen (produced by Chemexpress) as an active ingredient and polyethylene oxide (produced by Sumitomo Seika Chemicals Company, Limited; mass average molecular weight: 160,335 g/mol) as a binder were mixed in a mass ratio of 92.5:7.5 (active ingredient:binder) to prepare a mixture. 0.1% Aerosil (produced by Evonik Industries) was added to the mixture, followed by mixing using a blender (produced by Misugi Ltd., Mazemazeman SKH-40CA; 0.7 revolutions/sec) for 150 minutes or more, thereby obtaining a raw material for twin-screw extrusion.

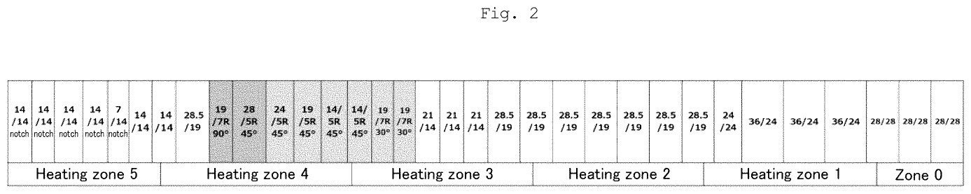

The obtained raw material was fed into a twin-screw extruder (TEM-18SS-10/2V; Toshiba Machine Co., Ltd.) equipped with screws shown in FIG. 2 at a feed rate of 1.8 kg/h by a gravimetric feeder (Kubota Keiso; model: CE-W-OE-MP), and granulation was performed under the conditions of a temperature of 25° C. in heating zone 1, a temperature of 30° C. in heating zones 2 to 5, and a screw speed of 200 rpm, to obtain granules. The twin-screw extruder used was composed of 10 barrels, and the length of each barrel was 75 mm. As shown in FIG. 2, the area of 0 to 75 mm from the feed port was designated as zone 0, the area of 75 to 225 mm from the feed port was designated as heating zone 1, the area of 225 to 375 mm from the feed port was designated as heating zone 2, the area of 375 to 525 mm from the feed port was designated as heating zone 3, the area of 525 to 675 mm from the feed port was designated as heating zone 4, and the area of 675 to 750 mm from the feed port was designated as heating zone 5. The heating temperature was controlled independently for each heating zone. The kneading part is located in heating zone 3 and heating zone 4 (shaded area in FIG. 2).

The obtained granules were pulverized in a food processor (Zojirushi; model: BM-RT08) (10 sec/time×3 times), and coarse particles were removed with a 32-mesh (mesh opening: 500 μm) sieve to obtain a preparation composition comprising granules (particles).

### Example 2

A preparation composition was obtained in the same manner as in Example 1, except that the temperature in heating zones 2 to 5 was 40° C.

### Example 3

A preparation composition was obtained in the same manner as in Example 1, except that the temperature in heating zones 2 to 5 was 50° C.

### Example 4

A preparation composition was obtained in the same manner as in Example 1, except that the temperature in heating zones 2 to 5 was 130° C.

### Example 5

A preparation composition was obtained in the same manner as in Example 1, except that the binder was polyethylene oxide (produced by Sumitomo Seika Chemicals Company, Limited; mass average molecular weight: 105,216).

### Example 6

A preparation composition was obtained in the same manner as in Example 1, except that the binder was polyethylene oxide (produced by Sumitomo Seika Chemicals Company, Limited; mass average molecular weight: 820,913).

### Comparative Example 1

A preparation composition was obtained in the same manner as in Example 1, except that the mass ratio of the active ingredient to the binder was 70:30 (active ingredient:binder).

### Comparative Example 2

A preparation composition was obtained in the same manner as in Example 1, except that the temperature in heating zones 2 to 5 was 150° C.

### Comparative Example 3

A preparation composition was obtained in the same manner as in Example 1, except that the temperature in heating zones 2 to 5 was 160° C.

### Evaluation Methods

### Measurement of Hardness of Preparation

The hardness of preparations was evaluated by measuring the compression strength of tablets. Specifically, 500 mg of the preparation composition (granules) obtained in each of the Examples and the Comparative Examples was taken, and a flat tablet (diameter: 10.7 mm, flat tablet) was produced at a tableting pressure of 10 kN with a tableting machine (Autograph AGS-J produced by Shimadzu Corporation). The tablet thickness was measured with a thickness gauge (Teclock Co., Ltd.). The hardness of the tablet was measured using a hardness tester (TBH325 tablet hardness tester produced by ERWEKA) under the following conditions: sensitivity: 10 N, measurement speed: 2.3 mm/s; measurement pressure: 20 N/s; measurement mode: speed. The hardness measured in this measurement was converted into compression strength using the following formula.

$$Compression strength [ MPa ] = \frac{Hardness [ kp ] \times 9.807 \times 2}{3.14 \times 10.7 [ mm ] \times Thickness [ mm ]}$$

### Measurement of Mass Average Molecular Weight of Polyethylene Oxide

The mass average molecular weight of polyethylene oxide was measured by gel permeation chromatography. Specifically, 0.02 g of polyethylene oxide was added and dissolved in 40 mL of a 0.19 M sodium nitrate aqueous solution over 3 hours, the resulting solution was filtered through a 0.8 μm membrane filter, and the obtained filtrate was subjected to measurement by gel permeation chromatography (HLC-8220GPC, produced by Tosoh Corporation; guard column: TSKgel guardcolumn PWXL). In this measurement, the size exclusion columns were TSKgel G6000PWXL, TSKgel GMPWXL, and TSKgel G3000PWXL, the mobile phase was a 0.20 M sodium nitrate aqueous solution, the flow rate was 0.5 mL/min, the column temperature was 40° C., the differential refractometer temperature was 40° C., the injection volume was 500 μL, and the measurement time was 90 minutes. Separately from this, the same measurement was carried out using a polyethylene oxide standard sample with known mass average molecular weight to create a calibration curve. Based on the calibration curve, the mass average molecular weight of polyethylene oxide was calculated.

### Particle Coverage

The coverage was measured by an ATR method (attenuated total reflection) using particles as a measurement sample. The same active ingredient and the same binder as the active ingredient (acetaminophen) and the binder (polyethylene oxide or hydroxypropyl cellulose) contained in a measurement sample were mixed in mass ratios (active ingredient:binder) of 25:75, 50:50, 75:25, 92.5:7.5, 95:5, and 97.5:2.5 to prepare six mixtures. The absorbance of each mixture was measured using a Fourier transform infrared spectrometer (JASCO Corporation), and a calibration curve was created from the ratio of the absorbance in an absorption band characteristic of the active ingredient and the absorbance in an absorption band characteristic of the binder. The absorption band of acetaminophen was selected at 1650 cm⁻¹, the absorption band of polyethylene oxide was selected at 2880 cm⁻¹, and a calibration curve was prepared. Subsequently, the absorbance of the particles as the measurement sample was also measured using a Fourier transform infrared spectrometer (JASCO Corporation), and the proportion of the binder present relative to the active ingredient and the binder was calculated by substituting the measured absorbance into the function for the calibration curve. This proportion was defined as the particle coverage.

### Granulation Properties

The granulation properties were visually determined. Specifically, the preparation composition obtained in each of the Examples was visually observed. For those in which excessive granulation was observed (i.e., having lumpy material that was difficult to pulverize) and those that remained in powder form, granulation was determined to be impossible. For the rest, granulation was determined to be possible. Cases in which it was determined that granulation was possible were evaluated as “A,” and cases in which it was determined that granulation was impossible were evaluated as “B.”

TABLE 1

| | | Binder | | | | | | | | | | |
|---|---|---|---|---|---|---|---|---|---|---|---|---|
| Example/ | Active | | Mass average | | Barrel temperature (° C.) | | | | | | | |
| Comparative | ingredient | | molecular | | Heating | Heating | Heating | Heating | Heating | Granulation | Coverage | Compression |
| Example | (mass %) | Type | weight (Mw) | Mass % | zone 1 | zone 2 | zone 3 | zone 4 | zone 5 | properties | (%) | strength (MPa) |
| Example 1 | 92.5 | PEO | 160,335 | 7.5 | 25 | 30 | 30 | 30 | 30 | A | 37.7 | 4.07 |
| Example 2 | 92.5 | PEO | 160,335 | 7.5 | 25 | 40 | 40 | 40 | 40 | A | 34.8 | 3.09 |
| Example 3 | 92.5 | PEO | 160,335 | 7.5 | 25 | 50 | 50 | 50 | 50 | A | 28.1 | 2.64 |
| Example 4 | 92.5 | PEO | 160,335 | 7.5 | 25 | 130 | 130 | 130 | 130 | A | 33.6 | 2.02 |
| Example 5 | 92.5 | PEO | 105,216 | 7.5 | 25 | 30 | 30 | 30 | 30 | A | 20.3 | 3.38 |
| Example 6 | 92.5 | PEO | 820,913 | 7.5 | 25 | 30 | 30 | 30 | 30 | A | 22.2 | 2.96 |
| Comparative | 70 | PEO | 160,335 | 30 | 25 | 30 | 30 | 30 | 30 | B | — | — |
| Example 1 | | | | | | | | | | | | |
| Comparative | 92.5 | PEO | 160,335 | 7.5 | 25 | 150 | 150 | 150 | 150 | A | 8.79 | 1.39 |
| Example 2 | | | | | | | | | | | | |
| Comparative | 92.5 | PEO | 160,335 | 7.5 | 25 | 160 | 160 | 160 | 160 | B | — | — |
| Example 3 | | | | | | | | | | | | |

Table 1 shows the production conditions for the preparation compositions produced in the Examples and the Comparative Examples (the proportions of the active ingredient and the binder and the temperature in each heating zone), the granulation properties of the preparation compositions, the particle coverage, and evaluation results of the compression strength of the tablets obtained from the preparation composition.

The results in Table 1 show that a preparation with excellent compression strength (i.e., with high hardness) can be obtained by using a preparation composition comprising particles that contains the active ingredient in an amount of 80 mass % or more and the binder in an amount of 20 mass % or less and that has a coverage of 20% or more.

1. A preparation composition comprising particles containing an active ingredient and a binder,
the particles containing the active ingredient in an amount of 80 mass % or more based on the total mass of the active ingredient and the binder, and
the coverage of the binder at the surface of the particles being 20% or more.

2. The preparation composition according to claim 1, wherein the binder is a water-soluble polymer.

3. The preparation composition according to claim 1, wherein the binder is at least one member selected from the group consisting of polyethylene oxide, hydroxypropyl cellulose, hydroxypropyl methylcellulose, polyvinylpyrrolidone, polymethacrylate, copolymers of N-vinylpyrrolidone and vinyl acetate, and copolymers of N-vinylcaprolactam, vinyl acetate, and ethylene glycol.

4. The preparation composition according to claim 1, wherein the binder has a mass average molecular weight of 1000000 g/mol or less.

5. The preparation composition according to claim 1, wherein the binder is polyethylene oxide having a mass average molecular weight of 50000 g/mol or more and 1000000 g/mol or less.

6. A preparation comprising the preparation composition according to claim 1.

7. A method for producing the preparation composition according to claim 1, the method comprising step 1 of kneading and granulating a mixture containing an active ingredient and a binder in barrels by a twin-screw extrusion method,
the temperature in each barrel being 130° C. or less,
the binder having a mass average molecular weight of 1000000 g/mol or less, and
the mixture containing the active ingredient in an amount of 80 mass % or more based on the total mass of the active ingredient and the binder.
